(19) United States
(12) Patent Application Publication
(10) Pub. No.: US 2007/0088326 A1
(43) Pub. Date: Apr. 19, 2007

(54) Catheter shaft connector

(75) Inventor: Kenneth C. Kennedy II, Clemmons, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 11/472,204

(22) Filed: Jun. 21, 2006

Related U.S. Application Data
(60) Provisional application No. 60/692,788, filed on Jun. 22, 2005.

(51) Int. Cl. A61M 39/10

(52) U.S. Cl. 604/533

(57) ABSTRACT

A medical device connector for connecting the shafts of two or more catheters, the connector being disposed nearer to the distal end of the combined length of the two or more catheters.

### CROSS-REFERENCE TO RELATED APPLICATIONS

This application claims priority to U.S. Provisional Application Ser. No. 60/692,788, filed Jun. 22, 2005, which is incorporated herein by reference in its entirety.

### BACKGROUND

The present invention is related to medical catheter devices, and more particularly to structures for connecting medical catheter devices to one another to form an elongate medical device.

In the field of medical catheters, a variety of connecting and joining technologies is well known as applied near the proximal end of catheter shafts. For example, connecting/joining technologies are used for forming luer hubs, dividing lumens from each other and/or access ports, merging lumens, marrying handles to catheter shafts and other such applications. These connecting/joining technologies have not been commonly applied in distal portions of catheter shafts. Although some joining technologies have been applied to attach dissimilar structures to the distal end of a catheter shaft (e.g., attaching inflation balloons to catheters, attaching a distal plastic catheter shaft to a proximal metal hypotube catheter shaft), there is not presently available a set of connectors readily usable for joining two or more catheter shafts co-linearly nearer to the distal end than to the proximal end of their combined length.

In certain applications, it is advantageous to longitudinally connect two catheter shafts having different physical properties. For example, in a medical procedure where a catheter is to be directed through an endolumenal passage, a proximal portion of the endolumenal passage may be fairly open, while a distal portion of the endolumenal passage may be small and tortuous. In such a circumstance it would be advantageous to have a catheter shaft with a stiffer proximal portion and a smaller, more flexible distal portion. The stiffer proximal portion provides for enhanced trackability and pushability of the catheter, while the smaller, more flexible distal portion provides for enhanced ability to navigate a narrow tortuous passage. There is a need for a catheter shaft connector that can efficiently connect catheter shafts having differing physical properties (e.g., stiffness, composition, diameter, outer profile/shape, or lumen number).

### BRIEF SUMMARY

The present invention provides a catheter shaft connector that can efficiently connect elongate shafts, such as shafts with a solid cross-section or lumenal catheter shafts, having different properties (e.g., differing in outside diameter; lumen size, number, or shape; or shaft stiffness). Thus, the above-mentioned needs are addressed in the present invention, which provides catheter shaft connecting structures for use in joining a distal end of a first catheter shaft to a proximal end of a second catheter shaft. Moreover, the present invention includes a connecting structure that, in some embodiments, provides for a port into an elongate catheter structure without diminishing the structural integrity of that elongate catheter structure.

In a first aspect, the present invention includes an elongate medical device with a connector body and first and second elongate shafts. The connector body includes a first connector end for union with an end portion of the first elongate shaft, a second connector end for union with an end portion of the second elongate shaft, and at least a first lumen extending between the first and second connector ends. The connector body preferably is disposed nearer a distal end than a proximal end of the united shaft that is formed when the first and second shafts are joined together. The first lumen provides a path of fluid communication between the first and second elongate shafts.

In a second aspect, the present invention includes a medical device structure for joining at least a first elongate shaft and a second elongate shaft. The medical device includes a body, a first end for union with one of the at least first and second elongate shafts, a second end for union with another of the at least first and second catheter shafts, and at least a first substantially solid dividing body between the first and second ends. The structure preferably is disposed nearer a distal end than a proximal end of the united shaft that is formed when the first and second elongate shafts are joined together.

In a third aspect, the present invention includes an elongate medical device connector with a connector body and first and second elongate shafts. The connector body includes a first connector end for union with an end portion of the first elongate shaft, a second connector end for union with an end portion of the second elongate shaft, and a solid central portion between the first and second connector ends. The connector body preferably is disposed nearer a distal end than a proximal end of the united shaft that is formed when the at least first and second shafts are joined together.

### BRIEF DESCRIPTION OF THE DRAWINGS

FIG. 1 shows one embodiment of a catheter connector;

FIG. 2 depicts another embodiment of a connector, including insertion prongs;

FIG. 3 shows a cross-sectional view of a solid-core connector;

FIG. 4 illustrates another embodiment of a connector;

FIG. 5 shows a cross-sectional view of a reducing connector;

FIG. 6 illustrates a cross-sectional view of a straight connector;

FIG. 7 depicts a cross-sectional view of another reducing connector;

FIG. 8 shows a cross-sectional view of a reducing connector with a straight outer profile;

FIG. 9 illustrates a cross-sectional view of a dual lumen connector;

FIG. 10 depicts a cross-sectional view of a merging/splitting lumen connector;

FIG. 11 shows a cross-sectional view of a narrowing lumen connector;

FIG. 12 depicts a cross-sectional view of a connector with a straight outer profile and a side port;

FIG. 13 illustrates a perspective view of an oblique side port connector;

FIG. 13A shows a cross-sectional view of the connector of FIG. 13;

FIG. 14 illustrates a cross-sectional view of another oblique side port connector;

FIG. 15 depicts a cross-sectional view of a reducing connector with a side port;

FIG. 16 illustrates a perspective view of a dual side port connector;

FIG. 16A depicts a cross-sectional view of the connector of FIG. 16A;

FIG. 17 shows a cross-sectional view of a connector with an inset oblique side port and a reducing lumen;

FIG. 18 shows a cross-sectional view of a reducing inset side port connector that includes an inset side aperture;

FIG. 19 illustrates a cross-sectional view of an application of an oblique port reducing connector; and

FIG. 20 depicts a perspective view of a catheter device including an oblique port connector.

### DETAILED DESCRIPTION

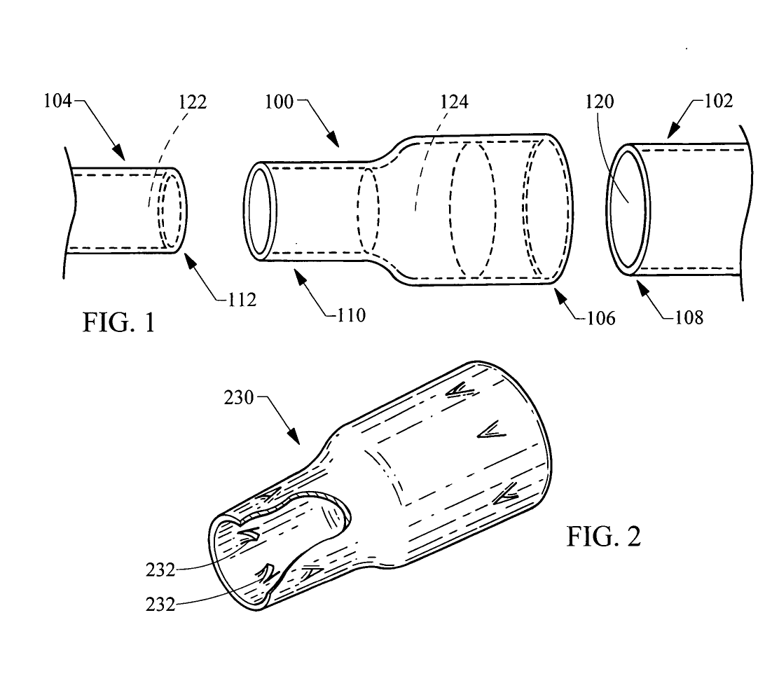

One embodiment of the present invention, illustrated in FIG. 1, provides a catheter connector 100 for connecting a first catheter shaft 102 with a second catheter shaft 104. Connectors of the present invention may be formed from any suitable material including, for example, metals, ceramics, polymers, and alloys, or combinations thereof. The first catheter shaft 102 has a greater diameter than the second catheter shaft 104. A larger end 106 of the connector 100 is sized to receive the distal end 108 of the first catheter shaft 102, and a smaller end 110 of the connector 100 is sized to receive the proximal end 112 of the second catheter shaft 104. The catheter shafts 102, 104 may be secured to the connector 100 by, for example, overmolding, heat staking, adhesive, crimping, or welding. Alternatively (or in combination with those securing means), as shown in FIG. 2, a connector 230 may include structures such as insertion prongs 232 that bitingly anchor the ends of catheter shafts (not shown) into the connector 230. Another securing means that may be used alone or in combination with one or more of the above is that a shrink tube or other overlay may be used to connect/secure a connector and adjacent shaft(s).

As illustrated in FIG. 1, the connector 100 provides for coupling the larger diameter catheter shaft 102 to the smaller diameter catheter shaft 104. The smaller catheter shaft 104 has a greater flexibility than the larger shaft 102 and therefore a greater ability to be navigated through small and/or tortuous passages. The larger first catheter shaft 102 provides greater columnar strength than the smaller catheter shaft 104, and therefore offers enhanced pushability and trackability. Combining the two catheter shafts 102, 104 longitudinally provides a combination of features advantageous for use in a branching endolumenal system of a body wherein proximal passages are more open and distal passages near a target site for the catheter device are less open and/or more tortuous. The catheters 102, 104 each have a central lumen 120, 122, respectively. When they are assembled to the connector 100, the lumens 120, 122 are in fluid communication with each other through a central lumen 124 of the connector 100.

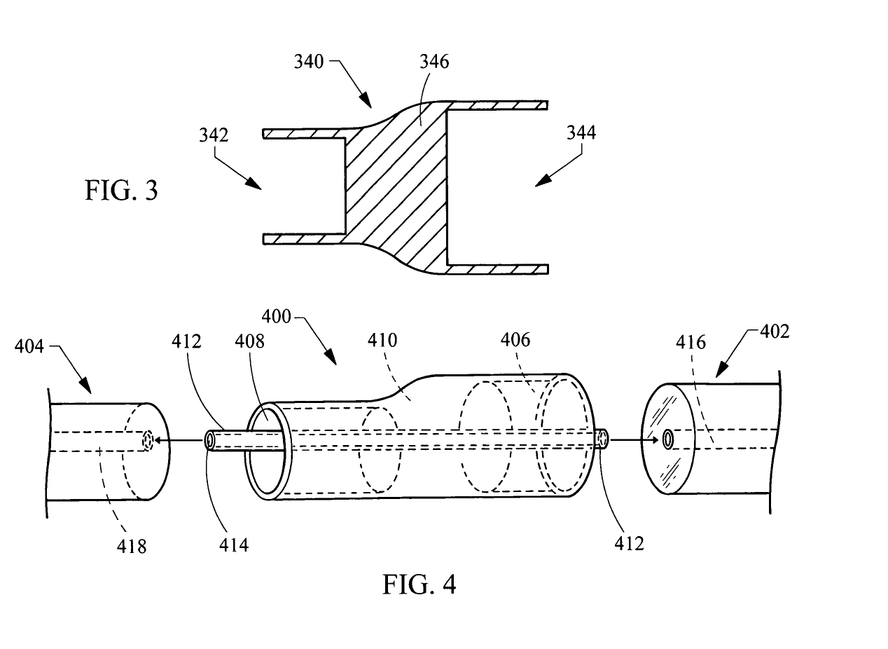

A cross-sectional view of an alternative connector embodiment, a solid-core connector 340, is illustrated in FIG. 3. The solid-core connector 340 has two shaft-receiving end lumens 342, 344 separated by a solid dividing core 346. A solid core connector 340 may be desirable in an application where a user who is connecting two or more catheters does not want fluid in a lumen of a distal catheter (such as, for example a short wire guide/rapid exchange type of catheter) to get past a connection to a proximal catheter, or vice versa. Alternatively, the solid core connector 340 may be useful in an application where a stylet or wire guide is introduced from the proximal end, and it is desirable to stop the distal movement of that stylet or wire guide at the connector 340.

Another embodiment of a connector of the present invention is shown in FIG. 4. The connector 400 joins a larger catheter shaft 402 to a smaller catheter shaft 404. The connector 400 has a larger lumen 406 at one end to receive the larger catheter shaft 402 and a smaller lumen 408 at the other end to receive the smaller catheter shaft 404. The catheter shafts 402, 404 each have a small lumen 416, 418, respectively. The middle portion 410 of the connector 400 is solid except for a cannula 412 having a lumen 414 extending therethrough. The ends of the cannula 412 extend through the larger and smaller lumens 406, 408 of the connector 400. When the larger catheter shaft 402 is mounted into the larger lumen 406, the cannula 412 aligns with and extends into the lumen 416 of the larger catheter shaft 402. Likewise, when the smaller catheter shaft 404 is mounted into the smaller lumen 408, the cannula 412 aligns with and extends into the lumen 418 of the smaller catheter shaft 404. The cannula lumen 414 provides a passageway of fluid communication between the larger catheter shaft's lumen 416 and the smaller catheter shaft's lumen 418. In addition, the cannula 412 may aid in aligning lumens when catheters are being joined.

The connector 400 is constructed of, for example, nitinol. In alternative embodiments, the connector 400 may be constructed of other materials such as other metallic alloys (e.g., stainless steel), a composite, polymers, ceramics, or a combination of these or other materials. The construction materials may be selected for particular characteristics such as, for example, a flexibility that is intermediate between the flexibilities of the catheter shafts being joined, or being formed of a composition that is, for example, moldable or heat-sealable to one or both of the catheter shafts being joined.

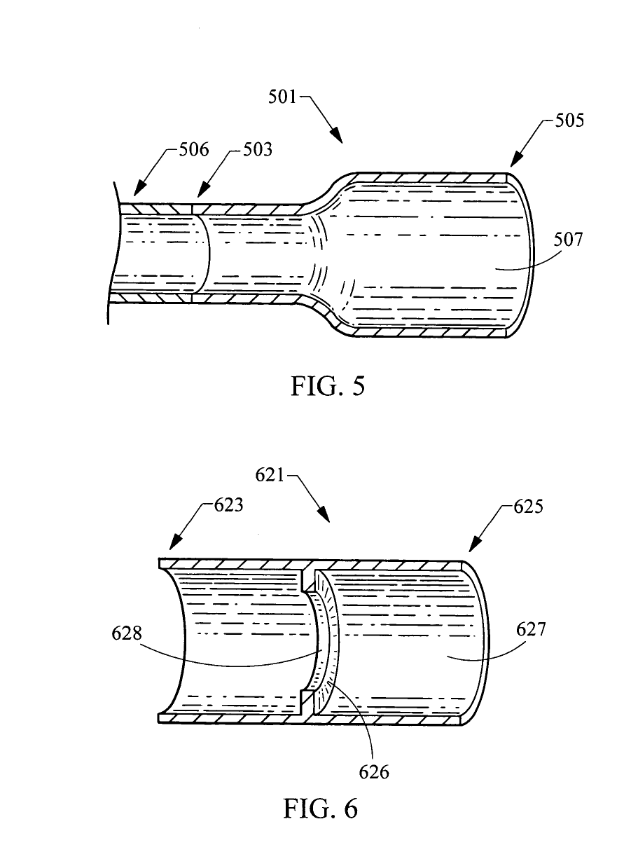

FIGS. 5-8 illustrate some different examples of connectors of the present invention having different exterior profiles, shapes, and cross-sections. FIG. 5 shows a first reducing connector 501. A smaller end 503 is centered axially relative to a larger end 505, and the connector 501 is configured for connecting a larger catheter shaft to a smaller catheter shaft. A lumen 507 extends through the length of the first reducing connector 501. FIG. 5 also illustrates that the connector 501 may be joined to a catheter 506 in an end-to-end configuration. The joint between the smaller end 503 of the connector 501 and the catheter 506 may be formed using, for example, a laser-weld, an adhesive, or some other joining mechanism.

FIG. 6 shows a first straight-outer-profile connector 621. Its ends 623, 625 are configured for connecting two catheter shafts of about the same size. A central protrusion 626 is in place in the central lumen 627 of the connector 621, and provides a surface for aligning and securing the ends of the two catheter shafts being joined. The opening 628 in the central protrusion 626 may be sized to correspond with the lumen size of the catheter shafts. In a preferred embodiment the opening 628 provides for a smooth transition between shaft lumens. Specifically, the shape of the central protrusion 626 and opening 628 preferably are sized and shaped at their proximal and distal ends to complement, respectively, the thickness and lumenal diameter of the catheter shafts.

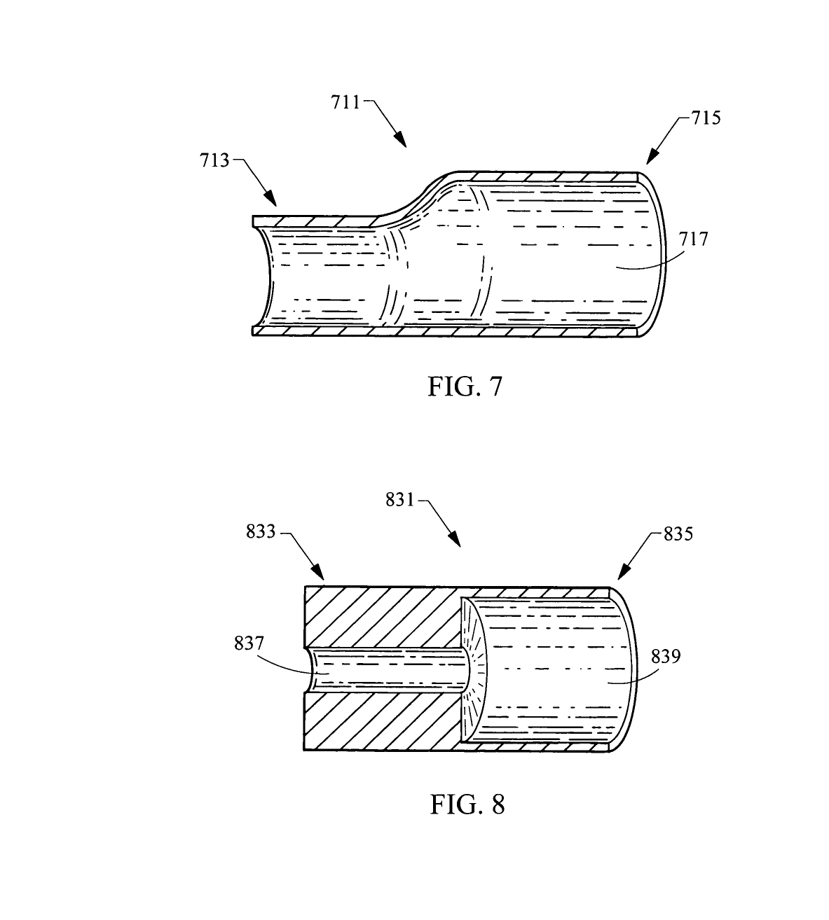

FIG. 7 shows a second reducing connector 711. A smaller end 713 is offset axially from center relative to a larger end 715, and the connector 711 is configured for connecting a larger catheter shaft to a smaller catheter shaft. A lumen 717 extends through the length of the second reducing connector 711. FIG. 8 shows a second straight-outer-profile connector 831 that is also a reducing connector. Its outer ends 833, 835 are about the same size. However, a small lumen 337 in the first end 833 has a lesser diameter than the large lumen 839 in the second end 835. The second straight-outer-profile connector 831 is configured for connecting a larger catheter shaft to a smaller catheter shaft, with the larger catheter shaft mounted into the large lumen 839 and the smaller catheter shaft mounted into the small lumen 837.

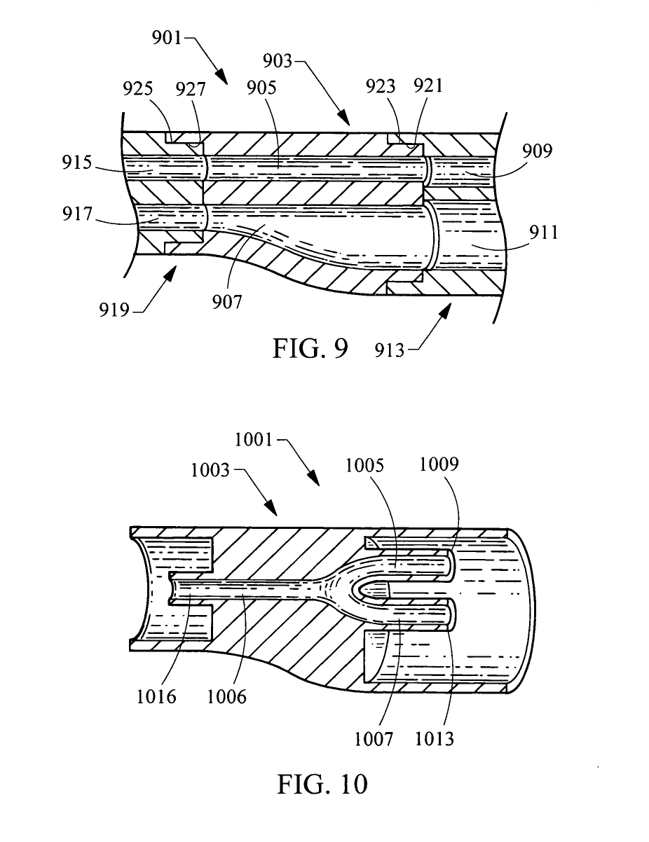

FIG. 9 depicts a cross-sectional view of a dual lumen reducing connector 901, with two catheters connected thereby to form an elongate medical device that has a smooth outer profile. The core region 903 of the connector includes two lumens 905, 907 for connecting two lumens 909, 911 of a larger proximal catheter 913 into fluid communication with two lumens 915, 917 of a smaller distal catheter 919. Of course, those of skill in the art will recognize that the proximal/distal positions of the catheters 913, 919 may be reversed. The connector 901 is configured to hold the catheters 913, 919 sealingly against the core region such that a path of fluid communication is provided from the first lumen 909 of the larger catheter 913 through the first connector lumen 905 to the first lumen 915 of the smaller catheter 919. Likewise, a path of fluid communication is provided from the second lumen 911 of the larger catheter 913 through the second connector lumen 907 to the second lumen 917 of the smaller catheter 919.

In order to provide a smooth outer profile of the overall elongate medical device comprising the catheters 913, 919 and the connector 901, the outer surface diameter of each of the catheters 913, 919 is the same as that of the connector end contacting the catheter. Also, the ends of each of the catheters 913, 919 are provided with shoulder surfaces configured to fittingly abut complementarily shaped grooved surfaces on the connector 901. Specifically, the distal end of the larger catheter 913 includes an inset shoulder 921 around its inner diameter. The inset shoulder 921 abuts to a complementarily shaped groove 923 around an end outer surface of the connector 901. The proximal end of the smaller catheter 919 includes an inset shoulder 925 around its outer diameter that complementarily abuts a groove 927 around the inner surface of the distal end of the connector 901. The effect of each of the complementarily shaped joining surfaces is that the elongate medical device created by connecting the two catheters 913, 919 has a substantially smooth profile across the portion where the catheters 913, 919 join and the outer diameter of the elongate medical device decreases across the connector 901, which has a partially tapered outer profile.

FIG. 10 illustrates a cross-sectional perspective view of a merging lumen reducing connector 1001. The core region 1003 of the merging lumen reducing connector 1001 is solid except for a pair of lumens 1005, 1007 that merge to form a single lumen 1006. The upper lumen 1005 extends through a first, upper cannula 1009. The upper cannula 1009 is configured to engage a lumen of a first catheter shaft (not shown). The lower lumen 1007 extends through a second, lower cannula 1013 that is configured to engage a lumen of a second catheter shaft (not shown). The merged single lumen 1006 extends through a cannula 1016 that is configured to engage a lumen of a third catheter shaft (not shown). Alternatively, the configuration of the upper and lower cannulas 1009, 1013 will allow them to engage, respectively, with two lumens of a multi-lumen catheter shaft (not shown).

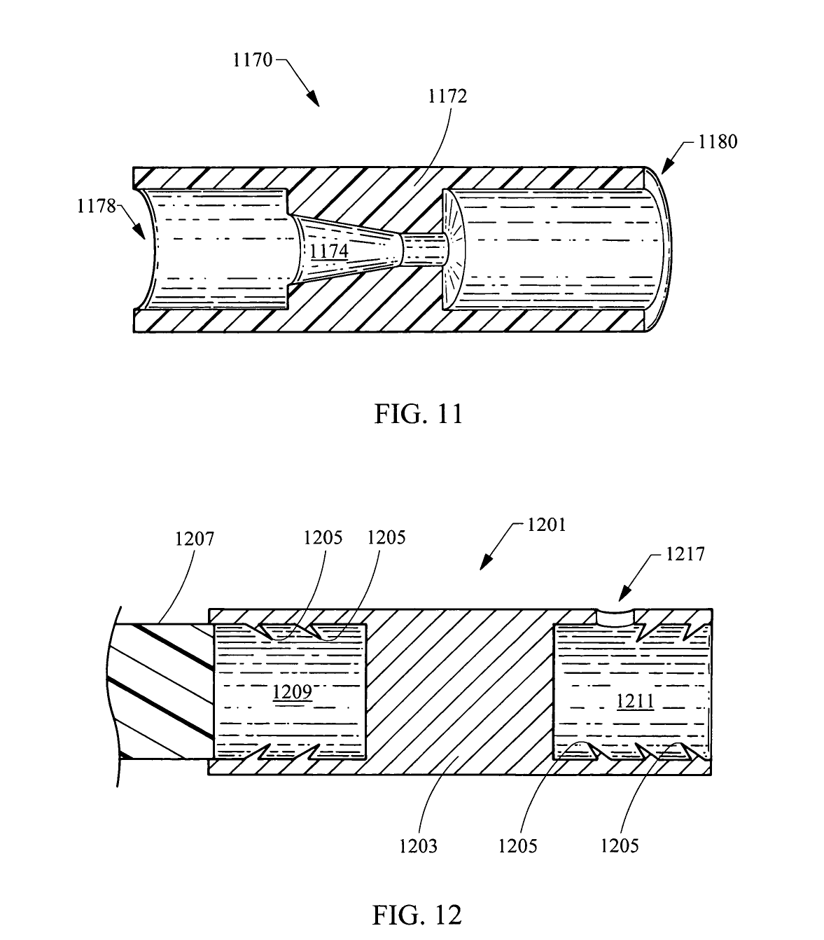

FIG. 11 illustrates a cross-sectional view of a narrowing lumen connector 1170 configured for connecting two catheter shafts (not shown) having substantially the same outer diameter, but having different inner/lumenal diameters. The core region 1172 of the single lumen connector 1170 is solid except for a reducing lumen 1174 extending therethrough. A first end 1178 of the connector 1170 is configured to engagingly receive an end of a first catheter shaft having a larger inner/lumenal diameter (not shown) and a second end 1180 of the connector 1170 is configured to engagingly receive an end of a second catheter shaft (not shown) having substantially the same outer diameter as the first catheter shaft, but having a smaller inner/lumenal diameter than the first catheter. In other embodiments, the catheter shafts may have outer diameters that differ from each other, and/or the inner diameters may be the same size. Alternatively, or in conjunction with the above-described use, the narrowing lumen connector 1170 may be used as a flow control device. Specifically, the diameter of the reducing lumen 1174 may be configured to control flow rate of a liquid passing therethrough.

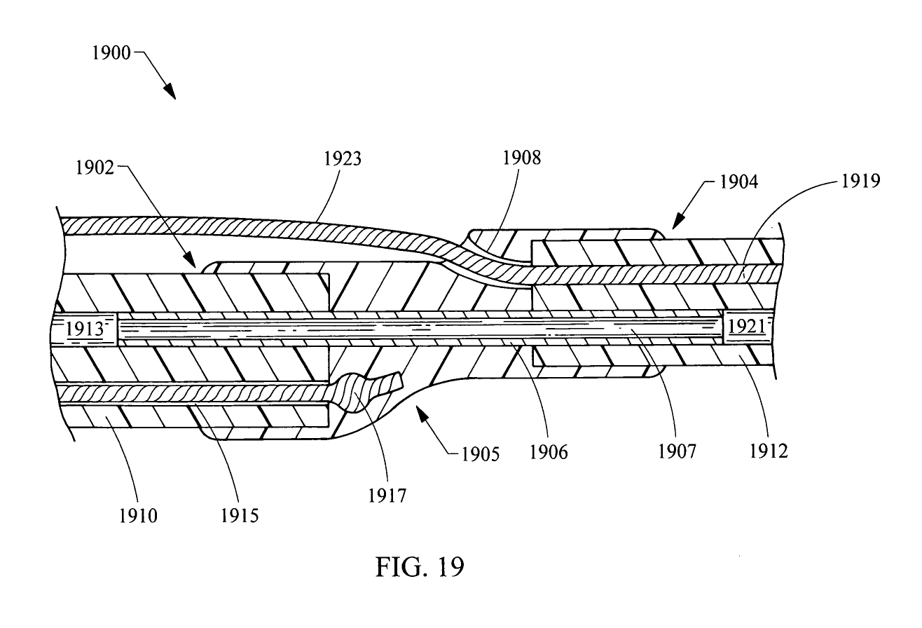

FIGS. 12-20 show embodiments of connectors of the present invention that each provide a side port. In these embodiments, the connector may provide structural support and/or integrity greater than that present in the region of a side port that is located within a wall of one of the catheters being connected with the connector. In particular, having a skived or other un-reinforced side port to provide access (e.g., for a wire guide in a short wire guide/rapid exchange application) tends to weaken the catheter in the region of the side port (e.g., the stiffness and/or strength of the catheter may be reduced). However, a side port introduced through a connector of the present invention allows the same access without significant deleterious effect on the strength of the catheter. Such a port can be located near either end or near the middle of the connector. Additionally, FIGS. 12 and 19 provide a ramped feature applicable to, for example, directing a wire guide to be used with one or both of the catheters being connected.

FIG. 12 is a cross-sectional view of a connector 1201. The central region of the connector 1201 is a solid core 1203. Barbs 1205 are provided to secure a first shaft 1207 into the proximal connector lumen 1209. As illustrated in the embodiment of FIG. 12, the first shaft 1207 is a solid “pusher-type” shaft. A side port 1217 is provided in a wall of the distal connector lumen 1211. The distal connector lumen 1211 also includes barbs 1205 to secure a second shaft (not shown) having a lumen. Similar barbs may also be useful in other embodiments. The shafts (707, second shaft not shown) may also be secured to the connector 1201 by other means such as an adhesive. The lumen of the second shaft may be used, for example, as a wire guide lumen. When the second shaft is placed into the distal connector lumen 1211, the side port 1217 provides an access path to the lumen of the second shaft.

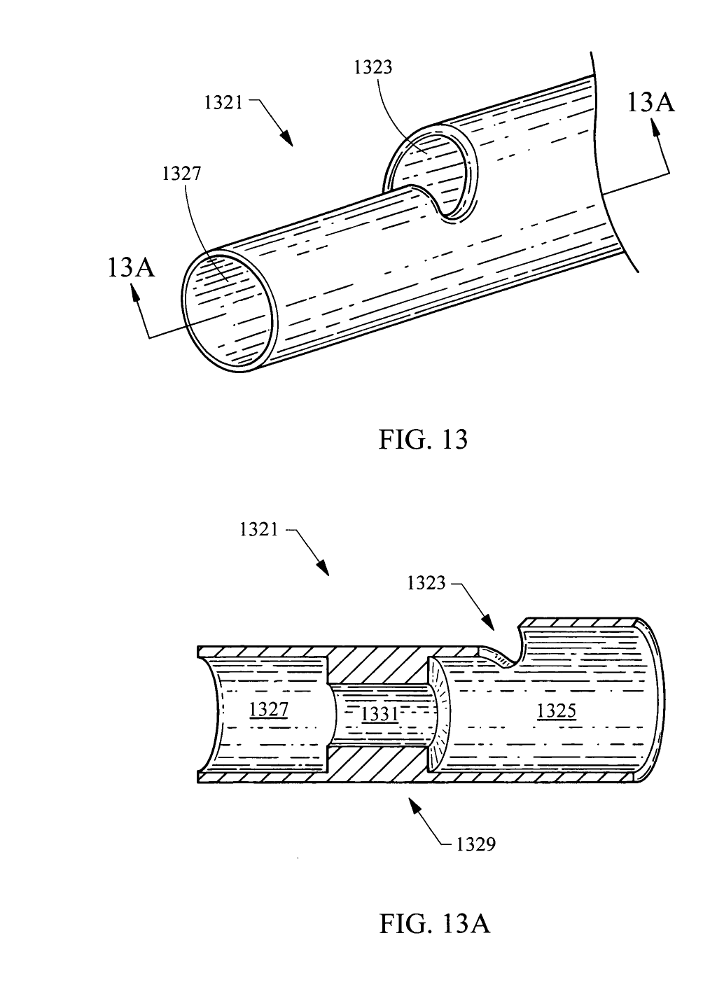

FIG. 13 shows a perspective view of an oblique side port connector 1321. FIG. 13A illustrates a cross-sectional perspective view of the oblique side port connector 1321 that includes an oblique side aperture 1323, which opens into a first connector lumen 1325. The first connector lumen 1325 is adapted to receive a larger catheter shaft than the second connector lumen 1327. The central portion 1329 of the connector 1321 includes a central lumen 1331, which provides a path of fluid communication between the first and second connector lumens 1325, 1327.

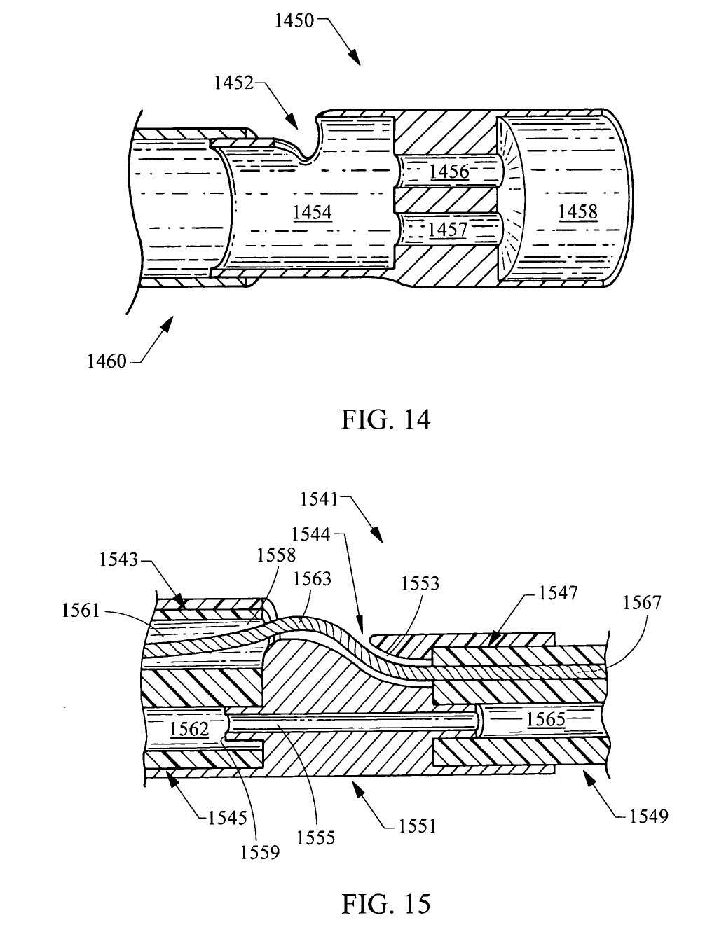

FIG. 14 depicts a cross-sectional view of an alternative embodiment of an oblique side port connector 1450 that includes a center-facing oblique side port 1452, which opens into a smaller connector lumen 1454. A pair of center lumens 1456, 1457 provides a fluid communication between the smaller connector lumen 1454 and a larger connector lumen 1458. A catheter 1460 is shown attached to the smaller end of the connector 1450. The attachment is effected by insertion of that smaller end into an end portion of the catheter 1460, where it may be secured by, for example, a frictional fit, laser weld, adhesive, or some other mechanism.

FIG. 15 depicts a cross-sectional perspective view of a central side port reducing connector 1541. The central side port reducing connector 1541 includes a larger first end lumen 1543 that is configured to receive a larger first catheter 1545 and a smaller second end lumen 1547 that is configured to receive a smaller second catheter 1549. The central region 1551 of the reducing connector 1541 is substantially solid except for two lumenal passages 1553, 1555. The first lumenal passage 1553 extends from a side aperture 1544 on the exterior of the central region 1551 of the connector 1541 to the second end lumen 1547. The second lumenal passage 1555 extends from the first end lumen 1543 through a cannula 1559 that protrudes through the central region 1551 of the connector 1541 and into the second end lumen 1547. The second lumenal passage 1555 through the cannula 1559 provides a path of fluid communication between the first and second end connector lumens 1543, 1547. An intermediate aperture 1558 is open from the first end lumen to the exterior of the connector 1541.

Additionally, FIG. 15 illustrates one application of the reducing connector 1541 wherein a larger first catheter 1545 is positioned in the first end lumen 1543. The larger first catheter 1545 is a dual lumen catheter with a lower catheter lumen 1562 and a wire guide lumen 1561 containing a wire guide 1563. The smaller second catheter 1549 is disposed in the second end lumen 1547. The smaller second catheter 1549 is a dual lumen catheter including a lower lumen 1565 and an upper lumen 1567. The two ends of the second passage cannula 1559 are engaged, respectively, into the lower catheter lumen 1562 of the larger first catheter 1545 and the lower lumen 1565 of the smaller second catheter 1549 such that fluid communication is provided therebetween. The upper lumen 1567 of the smaller second catheter 1549 is aligned with the first lumenal passage 1553.

As shown in FIG. 15 a wire guide 1563 extends from the wire guide lumen 1561 of the larger first catheter 1545 out through the intermediate aperture 1558, then into and through the side aperture 1544, the first lumenal passage 1553, and into the upper lumen 1567 of the smaller second catheter 1549. Alternatively, the reducing connector 1541 may be used in a short wire configuration (similar to that shown in FIG. 19). More specifically, the wire guide 1563 may extend only through the side aperture 1544, the first lumenal passage 1553, and into the upper lumen 1567 without passing through the intermediate aperture 1558 or the wire guide lumen 1561.

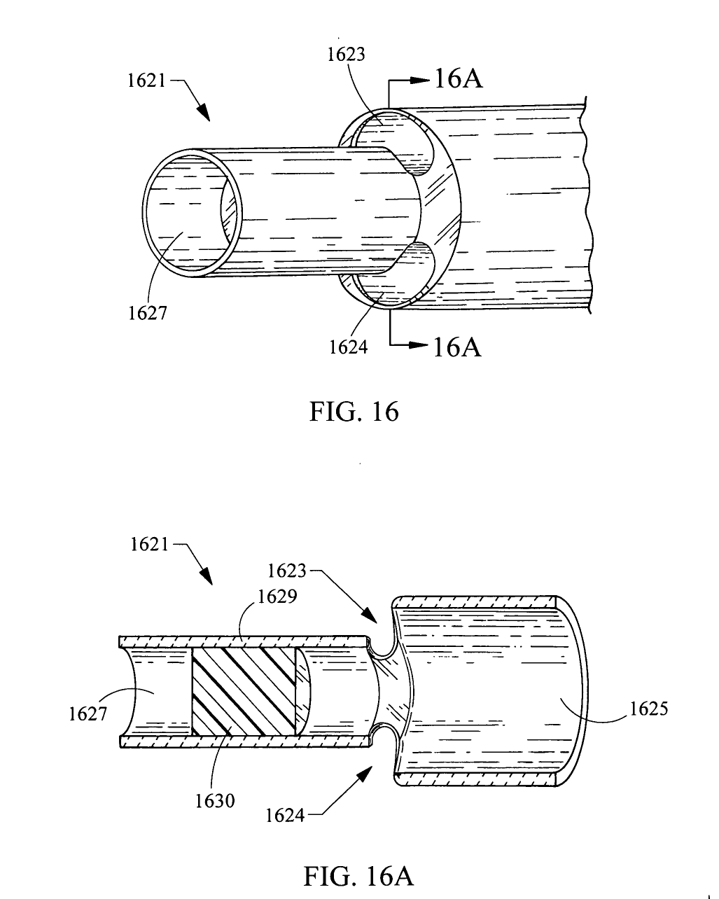

FIG. 16 shows a perspective view of a dual side port connector 1621. FIG. 16A illustrates a cross-sectional perspective view of the dual-side port connector 1621 that includes a pair of protruding side aperture structures 1623, 1624 which open into a first connector lumen 1625. The first connector lumen 1625 is adapted to receive a larger catheter shaft than the second connector lumen 1627. The central portion 1629 of the connector 1621 includes a solid center core body 1630 that separates the first and second connector lumens 1625, 1627.

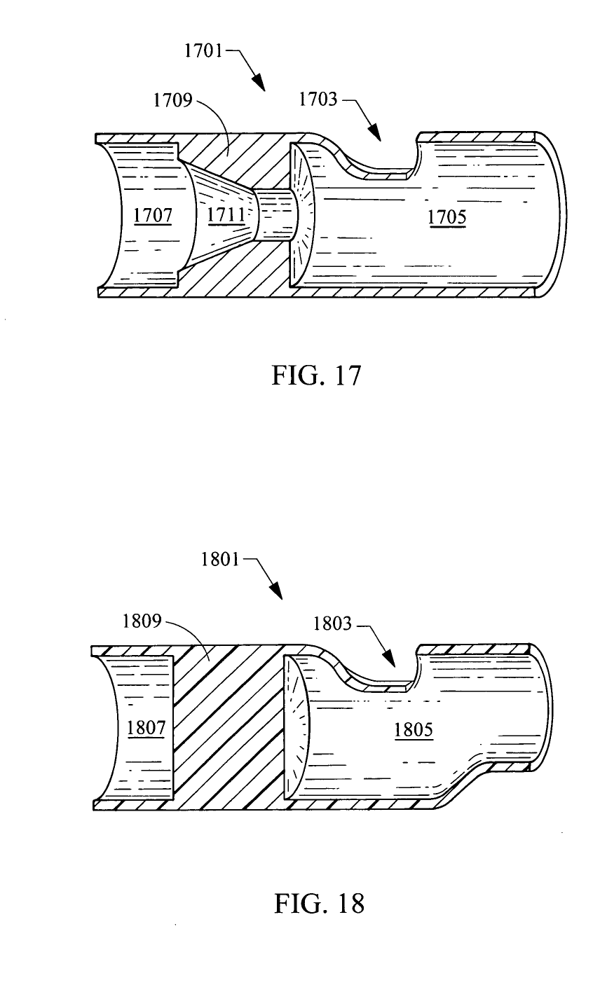

FIG. 17 depicts an inset-oblique side port connector 1701 that includes an inset oblique side aperture 1703 that opens into a first connector lumen 1705. The first connector lumen 1705 is separated from a second connector lumen 1707 by a central divider 1709 that includes a reducing lumen 1711 therethrough.

FIG. 18 shows a reducing inset side port connector 1801 that includes an inset side aperture 1803 that opens into a first connector lumen 1805. The first connector lumen 1805 is separated from a second connector lumen 1807 by a solid central divider 1809. A portion of the first connector lumen 1805 is smaller in diameter than the second connector lumen 1807 such that the first connector lumen 1805 is configured to engage a smaller catheter shaft than the second connector lumen 1807.

FIG. 19 illustrates one application of an oblique port reducing connector 1900. The connector 1900 includes a proximal mounting lumen 1902 and a distal mounting lumen 1904. The connector 1900 has a center portion 1905 that is substantially solid except for a cannula 1906 placed therethrough and an oblique side port 1908 that extends at angle from the side exterior of the connector 1900 to the distal mounting lumen 1904. The cannula 1906 includes a cannula lumen 1907, which provides a path of fluid communication through the center connector portion 1905. A larger diameter proximal catheter 1910 is disposed in the proximal mounting lumen 1902, and a smaller diameter distal catheter 1912 is disposed in the distal mounting lumen 1904. The proximal catheter 1910 is a dual lumen catheter with a first lumen 1913 and a second lumen 1915. The first lumen 1913 of the proximal catheter 1910 is engaged about a proximal portion of the cannula 1906. A wound cable stiffening member 1917 is mounted into the center connector portion 1905 and extends through the second lumen 1915 of the proximal catheter 1910. The smaller diameter distal catheter 1912 is also a dual lumen catheter, and has an upper lumen 1919 and a lower lumen 1921. The lower lumen 1921 is engaged about a distal portion of the cannula 1906 such that the lower lumen 1921 of the distal catheter 1912 is in fluid communication with the first lumen 1913 of the proximal catheter 1910 via the cannula lumen 1907. The upper lumen 1919 of the distal catheter 1912 is open to the oblique side port 1908. A wire guide 1923 is directed through the oblique side port 1908, into and through the upper lumen 1919 of the distal catheter 1912.

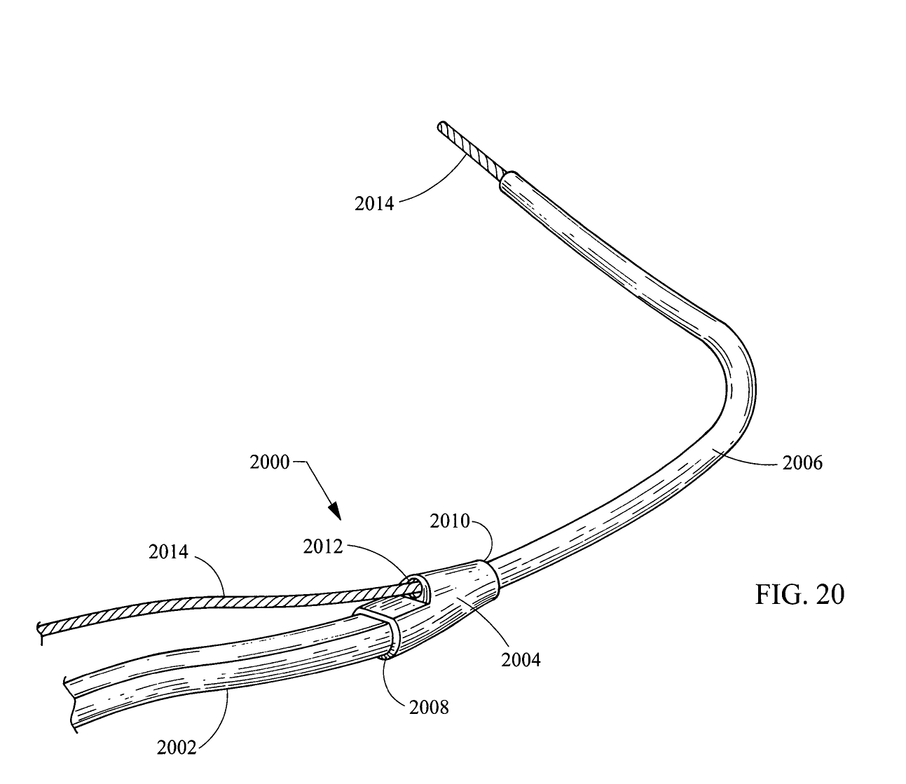

FIG. 20 depicts an elongate medical device 2000 including a proximal D-profile catheter shaft 2002, a connector 2004, and a distal round-profile catheter shaft 2006. The round-profile catheter 2006 has a smaller circumference and is more flexible than the D-profile catheter 2002. For the sake of illustration, the elongate medical device 2000 is shown in a shorter configuration than may typically be used. The connector 2004 includes a proximal end 2008 shaped to receive the D-profile catheter 2002 and a distal end 2010 shaped to receive the proximal end of the round-profile catheter 2006. The connector 2004 includes a side port 2012, which provides for introduction of a wire guide 2014 into the round profile catheter 2006. In alternative embodiments, the elongate device 2000 may include other catheter shafts having differently shaped profiles (e.g., V-shaped profile, crescent-shaped profile, or rectilinear profile) with the connector 2004 being configured to join those catheter shafts.

Various features and applications of the above-described embodiments may be interchangeable with each other or may be combined in ways not described herein without departing from the scope of the present invention. It is therefore intended that the foregoing detailed description be regarded as illustrative rather than limiting, and that it be understood that it is the following claims, including all equivalents, that are intended to define the spirit and scope of this invention.

We claim:

1. An elongate medical device, comprising:
a connector body; and
first and second elongate shafts;
wherein the connector body includes a first connector end for union with an end portion of the first elongate shaft, a second connector end for union with an end portion of the second elongate shaft, and at least a first lumen between the first and second connector ends;
wherein the connector body is disposed nearer a distal end than a proximal end of a united shaft that is formed when the at least first and second shafts are joined together; and
said first lumen providing a path of fluid communication between the first and second elongate shafts.

2. The device of claim 1, wherein the first connector end comprises a larger diameter than the second connector end.

3. The device of claim 1, further comprising an aperture through an exterior surface wherein said aperture is open to at least one of the first lumen, the first connector end, or the second connector end.

4. The device of claim 1, wherein the at least one of the first and second connector ends has an internal surface configured to fit over at least part of the end portion of the corresponding first or second elongate shaft.

5. The device of claim 4, further comprising insertion prongs in at least one of the first and second connector ends, wherein the insertion prongs are configured for anchoring a shaft disposed in one of the first and second connector ends.

6. The device of claim 4, further comprising mounting barbs in at least one of the first and second connector ends for anchoring a shaft disposed therein.

7. The device of claim 1, wherein the first elongate shaft comprises an exterior circumferential shape that is different than that of the second elongate shaft.

8. The device of claim 1, wherein the first connector end of the connector body and the end portion of the first elongate shaft comprise complementary shapes such that, when they are connected, the elongate medical device comprises a substantially smooth outer profile.

9. The device of claim 1, further comprising a cannula having first and second end apertures, said cannula disposed in the first lumen.

10. The cannula of claim 9, wherein a cannula length extending beyond the first connector end is sized to slidably enter a lumen of the first elongate shaft.

11. The device of claim 9, wherein at least one of the first and second cannula end apertures is open to an exterior surface of the device other than the first or second connector ends.

12. The device of claim 1, further comprising a mass formed in and occupying a substantial cross-section of the first lumen between the first and second connector ends, and having at least a first passageway therethrough.

13. The device of claim 12, further comprising a second passageway disposed through at least a portion of the mass.

14. The device of claim 13, wherein the second passageway merges with the first passageway.

15. The device of claim 12, wherein the passageway comprises at least one end aperture open through an exterior surface other than the first or second ends of the connector body.

16. The device of claim 12, wherein a cannula having first and second end apertures comprises the first passageway.

17. The device of claim 16, wherein a cannula length extending beyond the first connector end is sized to slidably enter a lumen of the first elongate shaft.

18. The device of claim 17, wherein the cannula length extending beyond the second connector end is sized to slidably enter a lumen of the second elongate shaft, thereby providing fluid communication between the lumen of the first elongate shaft and the lumen of the second elongate shaft.

19. The device of claim 1, wherein at least one of the first elongate shaft and the second elongate shaft comprises a catheter shaft, said catheter shaft having a catheter lumen extending longitudinally through at least a portion thereof.

20. A medical device structure for joining a first elongate shaft to a second elongate shaft, comprising:
a body;
a first end attached to a first elongate shaft;
a second end attached to a second elongate shaft;
a substantially solid dividing portion disposed between the first and second ends; and
the body being disposed nearer a distal end than a proximal end of a united shaft that is formed when the first and second elongate shafts are joined together.

21. An elongate medical device connector, comprising:
a connector body; and
first and second elongate shafts;
wherein the connector body includes a first connector end configured for union with an end portion of the first elongate shaft, a second connector end configured for union with an end portion of the second elongate shaft, a substantially solid central portion disposed between the first and second connector ends, and a lumenal passage open between the second connector end and an exterior surface of the connector body;
wherein the connector body is disposed nearer a distal than a proximal end of a united shaft that is formed when the at least first and second shafts are joined together.

22. The connector of claim 21, wherein the lumenal passage is disposed at least partially through the substantially solid central portion.
